(19) United States
(12) Patent Application Publication
(10) Pub. No.: US 2026/0256893 A1
(43) Pub. Date: Sep. 3, 2026

(54) PROPHYLAXIS OF NEONATAL ESCHERICHIA COLI SEPSIS

(71) Applicant: THE CHILDREN’S MERCY HOSPITAL, Kansas City, MO (US)

(72) Inventors: Susana CHAVEZ-BUENO, Kansas City, MO (US); Rachel CHEVALIER, Kansas City, MO (US)

(21) Appl. No.: 18/846,743

(22) PCT Filed: Mar. 14, 2023
(86) PCT No.: PCT/US2023/064276

§ 371 Date: Sep. 13, 2024

Related U.S. Application Data
(60) Provisional application No. 63/319,515, filed on Mar. 14, 2022.

(51) Int. Cl. A61K 38/40; A61K 9/00; A61P 31/04

(52) U.S. Cl. CPC A61K 38/40; A61K 9/0034; A61P 31/04

(57) ABSTRACT

Methods and formulations to decrease bacterial loads in a neonate to prophylactically treat neonatal E. coli infection during pregnancy, comprising maternal administration of lactoferrin vaginally during the second half of pregnancy before birth of the neonate. Prophylactic vaginal formulations for maternal administration to decrease bacterial loads in a neonate and inhibit neonatal E. coli invasive infection including sepsis, comprising lactoferrin dispersed or suspended in a pharmaceutically acceptable carrier. Use of lactoferrin to prepare maternal vaginal medicaments, and use of lactoferrin for maternal vaginal administration during the second half of pregnancy to prophylactically treat neonatal E. coli infection.

### CROSS-REFERENCE TO RELATED APPLICATIONS

The present application claims the priority benefit of U.S. Provisional Patent Application Ser. No. 63/319,515, filed Mar. 14, 2022, entitled MATERNAL ANTEPARTUM ADMINISTRATION OF LACTOFERRIN FOR PREVENTION OF NEONATAL ESCHERICHIA COLI SEPSIS, incorporated by reference in its entirety herein.

### BACKGROUND OF THE INVENTION

### Field of the Invention

The present invention relates to preventative measures against bacterial illness in neonates, and in particular, maternal lactoferrin administration preceding labor and delivery to ameliorate prenatally acquired invasive neonatal E. coli infection.

### Description of Related Art

Newborn babies are at risk of acquiring infections from pathogenic agents in utero, even from microorganisms that may normally reside in the pregnant patient's reproductive system. Intrauterine infection is a major cause of premature labor with and without intact membranes. Neonatal invasive infections, including sepsis, often present in the hours following birth, and are associated with acquisition of microorganisms from the pregnant patient, which can occur through an ascending infection from an infected pregnant patient or may be acquired by the neonate as it passes through the birth canal during labor and delivery. The microorganisms most commonly associated with early-onset infection include Group B Streptococcus (GBS), Escherichia coli, and Staphylococcus. GBS infection has led to the widespread adoption of prenatal screening for GBS and treatment protocols. When screening shows that the pregnant patient is GBS positive, antibiotics to prevent against GBS infection are administered to the pregnant patient right before birth. However, antibiotics for GBS are not routinely active against common strains of E. coli that infect newborns and are a major cause of neonatal bacterial invasive infections and sepsis. Moreover, this frequent administration of antibiotics has led to concerns regarding the widespread antibiotic use in pregnant patients and newborns that can lead to potential adverse health outcomes.

E. coli is the most common cause of neonatal early-onset sepsis (i.e., invasive infections occurring within 72 hours of birth) and a leading cause of neonatal meningitis. Vaginal pathogenic E. coli strains ascend into the pregnant uterus infecting the offspring of colonized pregnant patients, or are acquired by the neonate as it passes through the birth canal during labor and delivery. Alarmingly, no preventative strategies against newborn E. coli sepsis exist. There are no vaccines, antibiotics, or any other measures that are currently used in pregnant patients to prevent neonatal E. coli invasive disease. Instead, current approaches are limited to treatment after infection, using antibiotics and other medications given to the infant after they have presented with signs of infection. Depending on the severity of the infection and the fragility of the newborn, these methods are often not effective, leading to a high incidence of adverse outcomes including developmental delays, hearing impairments, abnormal head growth and even death. Affected infants have up to 40% chance of dying from sepsis. Globally, the WHO estimates that 340,000 newborns die of sepsis annually.

Lactoferrin (LF) is an antibacterial and immunomodulatory glycoprotein that has been given to preterm newborns after birth to address late-onset sepsis. Vaginal administration of lactoferrin in pregnancy has also been used in the first trimester to decrease rates of preterm birth in clinical trials. However, the effect of lactoferrin in preventing E. coli spread from the pregnant patient to the newborn, reducing bacterial loads in the newborn, and for preventing E. coli sepsis in the newborn has not been investigated.

### SUMMARY

As previously explained, neonates are at risk of bacterial infection, for example when E. coli ascends through the birth canal infecting the fetus and resulting in early-onset neonatal invasive infection, e.g., sepsis that presents within hours after birth. Thus, described herein are methods and formulations for maternal lactoferrin administration as a prophylaxis to mitigate neonatal E. coli invasive infection, particularly by clinical isolates that cause sepsis, meningitis, developmental delays, and other adverse health outcomes in newborns. The lactoferrin formulation is administered prophylactically to the pregnant patient, potentially beginning mid-gestation, and in any case in the weeks, days or hours leading up to labor and delivery to protect the newborn against infection, particularly against invasive infection. As used herein, references to “invasive” infection refer to a (usually severe) internal infection of the neonate, such as in the blood, cerebrospinal fluid, organs, organ cavities, and the like, which often lead to sepsis and/or meningitis. Infections can also be “noninvasive” such as infections of the skin, eyes, or ears, for example, but are not usually as severe or resistant to treatment as an invasive bacterial infection.

In one aspect, embodiments described herein concern methods of decreasing bacterial loads in neonates, for example to inhibit neonatal E. coli infections. The methods generally comprising vaginal administration of a therapeutically-effective amount of lactoferrin to a pregnant mammal before birth of the neonate, and preferably at any point after the midpoint of gestation (i.e., from 20 weeks or later in a human patient). Advantageously, the bacterial load (e.g., of E. coli) in the neonate is reduced after birth, as compared to an untreated control. Preferably, the bacterial load is reduced by at least about 25%, preferably at least about 50%, and more preferably by at least about 75%, as compared to an untreated control, where the term “bacterial load” is defined to mean the number of bacteria present in an infected area.

Also described herein are prophylactic vaginal formulations comprising lactoferrin in a pharmaceutically-acceptable carrier for decreasing bacterial loads in a neonate, for example, to inhibit neonatal E. coli invasive infection including sepsis.

Uses of the formulations for prophylaxis against neonatal bacterial infections via maternal administration are also described herein.

### BRIEF DESCRIPTION OF THE DRAWINGS

FIG. 1 is a graph of the growth curves comparing wild-type RS218 and RS218-Cam^{R}.

FIG. 2 is a graph comparing the vaginal RS218-Cam^{R} loads in LF-pretreated pregnant mice compared to placebo-treated dams.

FIG. 3 is a graph comparing the placental RS218-Cam^{R} loads in hLF-pretreated mice compared to placebo-treated dams.

FIG. 4 is a graph comparing the bacterial loads in the blood of embryos of hLF-pretreated dams compared to those from placebo-treated dams.

FIG. 5 is a graph comparing the bacterial loads in the liver and spleen of embryos of hLF-pretreated dams compared to those from placebo-treated dams.

FIG. 6 is a graph comparing the bacterial loads in the brain of embryos of hLF-pretreated dams compared to those from placebo-treated dams.

FIG. 7 shows graphs of (A) the bacterial loads in the liver and spleen of embryos of bLF-pretreated dams compared to those from placebo-treated dams; and (B) the bacterial loads in the blood of embryos of bLF-pretreated dams compared to those from placebo-treated dams.

FIG. 8 shows graphs of (A) the bacterial loads in the placenta of embryos of bLF-pretreated dams compared to those from placebo-treated dams; and (B) the bacterial loads in the brain of embryos of bLF-pretreated dams compared to those from placebo-treated dams.

FIG. 9 shows graphs of (A) the bacterial loads in the liver and spleen of embryos of bLF-pretreated dams compared to those from placebo-treated dams; and (B) the bacterial loads in the blood of embryos of bLF-pretreated dams compared to those from placebo-treated dams.

FIG. 10 shows graphs of (A) the bacterial loads in the placenta of embryos of bLF-pretreated dams compared to those from placebo-treated dams; and (B) the bacterial loads in the brain of embryos of bLF-pretreated dams compared to those from placebo-treated dams.

FIG. 11 is a graph comparing the vaginal RS218-Cam^{R} loads in bLF-pretreated pregnant mice compared to placebo-treated dams.

### DETAILED DESCRIPTION

As described herein, newborns are at risk of becoming infected with E. coli and other microorganisms, particularly bacteria, including those that may normally reside in the intestine and genital tract of pregnant patients (even without causing symptoms of infection in the pregnant patient). Medical providers currently have no standard of care to prophylactically address neonatal E. coli invasive infection that can lead to sepsis, meningitis, and other adverse health outcomes.

In more detail, described herein are methods and formulations for prophylactic treatment of bacterial infections, particularly E. coli infections, in neonates via maternal administration of lactoferrin. The methods and formulations can be used to prevent or inhibit the spread of bacterial infection from the pregnant patient to the newborn. The methods and formulations can be used to decrease neonatal bacterial loads. The methods and formulations can be used to reduce the risk or severity of neonatal infection, sepsis, meningitis, and/or death from bacterial infection. The methods and formulations described herein for vaginal administration of lactoferrin to the pregnant patient during pregnancy (i.e., before birth of the neonate) will reduce the risk and adverse outcomes of bacterial infection in the neonate and improve the health outcomes after birth.

The methods and formulations are described herein as prophylactic approaches and preventative measures. In other words, the formulations are administered in advance to protect the neonate from illness, disease, symptoms, or other morbidity of the infection. Thus, the terms “prophylactic,” “prophylaxis,” “prevention,” and the like are specifically defined herein to refer to treatments, approaches, and formulations that are intended to inhibit, reduce, mitigate, ameliorate, or lesson the effects of a (future) infection to which the neonate may become exposed (but is not currently infected with). In some cases, the methods and formulations may prevent the development of observable morbidity from the bacterial infection (i.e., near 100% prevention). In other cases, the methods and formulations may only partially prevent and/or lessen the extent of morbidity due to the bacterial infection, in that severity of the symptoms and/or effects of the infection, and/or the duration of the infection/symptoms/effects is reduced, but the neonate may still become symptomatic, although the infection is not invasive and/or severe in that bacterial loads detected in the blood and/or cerebrospinal fluid are low. In some cases, the methods and formulations protect the neonate from severe infection, including sepsis, and/or reduce the incidence of adverse outcomes from bacterial infection, such as meningitis, developmental delays, hearing impairments, abnormal head growth, and even death. In any case, the methods and formulations are still considered to “prophylactic” or “preventative” of a potential bacterial illness. Conversely, non-prophylactic “treatments” or methods for merely “treating” and the like are used herein to refer to approaches and formulations intended to induce a beneficial change in an existing condition (e.g., bacterial infection) of a subject after the subject has already developed symptoms of the infection. For avoidance of doubt, the methods and formulations described herein are expressly aimed at preventative measures, which are administered before a subject has developed signs or symptoms of the infection (and in any case before birth of the neonate). In neonates, initial symptoms of infection might be few (or even absent, even in severe infections), but can include irregular heart rate (high or low) and/or irregular respiratory rate/apnea (high or low). Other signs of infection include poor perfusion (as pallor and/or mottled skin), hypothermia, lethargy, and/or hypoxia. Fever can also be symptom but is very rare in neonates.

The methods and formulations are intended for use by providers caring for pregnant patients in an outpatient or inpatient setting. As the pregnancy progresses, lactoferrin would be administered vaginally to the pregnant patient at any time after the midpoint of gestation (halfway point of gestation), which for human patients is 20 weeks. That is, the methods and formulations are intended for use during pregnancy (i.e., before birth of the neonate), and preferably during the second half of pregnancy, more preferably after week 25 of gestation for humans, and even more preferably in the third trimester for humans, and more particularly for any pregnant patient, during the days or weeks leading up to labor and delivery. The methods and formulations are effective at protecting the neonate against illness from various bacteria including neonatal E. coli strains similar to RS218 and SCB34, other extra-intestinal pathogenic E. coli (ExpEC) strains, and other neonatal gram-negative and gram-positive neonatal pathogens including group B Streptococcus, and the like.

The formulations generally comprise lactoferrin dispersed or suspended in a suitable pharmaceutically-acceptable carrier. The term carrier is used herein to refer to diluents, excipients, vehicles, and the like, in which the lactoferrin may be dispersed or suspended for administration. Suitable carriers will be pharmaceutically acceptable. As used herein, the term “pharmaceutically acceptable” means not biologically or otherwise undesirable, in that it can be administered to a subject without excessive toxicity, irritation, or allergic response, and does not cause unacceptable biological effects or interact in a deleterious manner with any of the other components of the composition in which it is contained. A pharmaceutically-acceptable carrier would naturally be selected to minimize any degradation of the lactoferrin and to minimize any adverse side effects in the subject, as would be well known to one of skill in the art. Pharmaceutically-acceptable ingredients include those acceptable for veterinary use as well as human pharmaceutical use.

Preferably, the carrier is selected to facilitate the deposition or application of the lactoferrin in the vaginal mucosa, and more preferably, help prolong its stability and efficacy duration in the applied milieu. For example, carriers suitable for mucosal administration, particularly vaginal administration are used. Exemplary carriers include polyacrylic acid polymers and derivatives (e.g., Carbopol® polymers, including acrylic acid crosslinked with allyl sucrose or allyl pentaerythritol, acrylic acid and C10-C30 alkyl acrylate crosslinked with allyl pentaerythritol, and interpolymers such as carbomer homopolymer or copolymer that contains a block copolymer of polyethylene glycol and a long chain alkyl acid ester), polymethacrylate copolymers (e.g., Eudragit®), polylactic-co-glycolic acid, guar gum, polymeric emulsifiers (e.g., Pemulen™, high molecular weight copolymers of acrylic acid and C10-C30 alkyl acrylate crosslinked with allyl pentaerythritol), polycaprolactone, polyethylenimine, glycerin, hyaluronic acid, ethylcellulose, Hypromellose (hydroxypropyl methyl cellulose), sodium carboxymethylcellulose, propylene glycol, sodium alginate, chitosan, polyvinyl alcohol, gelatin, mineral oil, petrolatum, solid fats and oils (e.g., coconut oil), polyvinyl pyrrolidone, mixtures thereof, copolymers thereof, hydrogels thereof, and crosslinked matrices thereof.

The carrier (with lactoferrin) is formulated for targeted delivery to mucosal tissues. In one or more embodiments, the carrier is a viscous solution in which the lactoferrin is dispersed or suspended. In one or more embodiments, the carrier is a hydrogel in which the lactoferrin is dispersed or suspended. In one or more embodiments, the carrier is a thin film membrane in which the lactoferrin is dispersed or suspended. In one or more embodiments, the carrier is a cream, lotion, salve, or ointment in which the lactoferrin is dispersed or suspended. In one or more embodiments, the carrier is a solid, semi-solid, or meltable suppository in which the lactoferrin is dispersed or suspended. In one or more embodiments, the carrier is a nanoparticle or microparticle. In one or more embodiments, the lactoferrin is encapsulated. Other ingredients may be included in the composition, such as other prebiotics, as well as probiotics, antiseptics, antibiotics, analgesics, immunomodulators, and the like. In one or more embodiments, the composition is essentially free of any other active agents, and consists essentially or even consists of lactoferrin in a mucosally-appropriate carrier.

The composition can comprise a therapeutically effective amount of lactoferrin dispersed in the carrier. As used herein, a “therapeutically effective” amount refers to the amount administered to the pregnant patient that will elicit the biological or medical response of a tissue, system, or subject that is being sought by a researcher or clinician, and in particular elicit some desired prophylactic effect as against the bacterial infection by inhibiting bacterial spread and decreasing bacterial load in the neonate (as compared to an untreated control). One of skill in the art recognizes that an amount may be considered therapeutically “effective” even if infection is not entirely prevented, but it or its symptoms and/or effects are improved or alleviated in the neonate (namely that invasive bacterial loads in the offspring are significantly decreased such that severe illness is prevented, where “significance” is defined herein as a p-value <5%). In one or more embodiments, the bacterial load in the offspring is reduced by at least 25%, preferably by at least 50%, and more preferably by at least 75% as compared to an untreated control. In one or more embodiments, the reductions refer to the reduction in bacterial load as detected in a blood sample from the neonate.

Notably, an amount is considered “therapeutically effective” even if it does not significantly reduce the bacterial load in the pregnant patient directly; rather, as demonstrated via the working examples, the protective effect in the offspring is nonetheless obtained via reduction in bacterial load in the neonate, even where the pregnant patient's levels of bacteria even in the vaginal milieu are not reduced. Thus, a “therapeutically effective” amount is not one that necessarily reduces bacterial load in the pregnant patient. Rather, it is the reduction of bacterial load in the offspring that is the objective. Without wishing to be bound by theory, it is hypothesized that the lactoferrin inhibits the ability of the bacteria to spread and proliferate to the offspring. Therefore, the aim is not to necessarily reduce or inhibit bacterial load in the pregnant patient herself, but instead interfere with the bacteria's ability to infect the neonate. As noted above, many microorganisms may normally reside in the pregnant patient without causing infection, but may nonetheless be deleterious to the neonate's more fragile immune system. In one or more embodiments, the composition comprises from about 100 μg/mL (0.1 mg/mL) lactoferrin to about 100 mg/mL lactoferrin, preferably from about from about 100 μg/mL to about 10 mg/mL lactoferrin, preferably from about 100 μg/mL to about 1,000 μg/mL (1 mg/mL) lactoferrin for liquid or gel formulations. In a solid formulation, the composition of lactoferrin in relation to vehicle will be 0.1 mg to about 100 mg of lactoferrin per gram of vehicle.

In one or more embodiments, the lactoferrin formulation can be provided in unit dosage form in a suitable container. The term “unit dosage form” refers to a physically discrete unit suitable as a unitary dosage for human or animal use. Each unit dosage form may contain a predetermined amount of lactoferrin in the carrier calculated to produce the desired effect. In other embodiments, the lactoferrin can be provided separate from the carrier (e.g., in its own vial, ampule, sachet, or other suitable container) for on-site mixing and preparation before administration to a subject. A kit comprising the lactoferrin is also disclosed herein. The kit further comprises instructions for administering the lactoferrin to a subject. The lactoferrin can be provided as part of a dosage unit, already dispersed in a pharmaceutically-acceptable carrier, or it can be provided separately from the carrier. The kit can further comprise instructions for preparing the lactoferrin for administration to a subject, including for example, instructions for dispersing the lactoferrin in a suitable carrier. The lactoferrin itself can be provided as a liquid for a powder form. Human or bovine lactoferrin can be used. Recombinant lactoferrin can also be used.

In use, the lactoferrin formulation is administered vaginally to a pregnant patient before labor and delivery/birth of the newborn. It is foreseen that the lactoferrin can additionally be used in instances of preterm labor. Preferably, the lactoferrin formulation is administered during the second half of gestation, more preferably during the week immediately preceding birth (i.e., during about 7 days before birth of the neonate). In one or more embodiments, the lactoferrin formulation is administered to the pregnant patient at least once daily (i.e., every 24 hours). In one or more embodiments, the lactoferrin formulation is administered daily for up to seven days (before labor begins or before delivery of the newborn). In one or more embodiments, the lactoferrin formulation is administered beginning seven days before the calculated due date. In one or more embodiments, the lactoferrin formulation is administered twice daily. In one or more embodiments, the lactoferrin formulation is administered 24 hours or less before onset of labor in said pregnant patient. In one or more embodiments, the lactoferrin formulation is administered at least 12 hours before onset of labor, preferably at least 24 hours before onset of labor. In one or more embodiments, the lactoferrin formulation is administered between about 12 and 24 hours before onset of labor. In one or more embodiments, the lactoferrin formulation is administered within 48 hours preceding the onset of labor in said pregnant patient, or at least within 48 hours before birth of the neonate. In one or more embodiments, the lactoferrin formulation is administered at least once after onset of labor in said pregnant patient and before birth/delivery of the newborn. Notwithstanding the foregoing, it is envisioned that a delayed-release formulation could be administered to the patient at the desired timepoint discussed above before labor and delivery, wherein it will provide a delayed or timed-release dosage of lactoferrin over a period of several hours, days, weeks, etc., depending on the formulation. For example, it is envisioned that a delayed-release form could be administered weekly or every few days starting from the midpoint of gestation until labor and delivery to provide a continual release of lactoferrin to the pregnant patient.

In one or more embodiments, the lactoferrin formulation is administered vaginally by applying the formulation to the mucosal membranes in the vagina of the pregnant patient. In one or more embodiments, the lactoferrin formulation is administered by insertion of a bolus, hydrogel, thin film, or suppository of the formulation into the vagina of the pregnant patient. In one or more embodiments, the lactoferrin formulation is administered in a dosage amount and frequency that maintains greater than about 10 μg/mL lactoferrin in the vaginal fluid of said pregnant patient. It will be appreciated that prophylactic methods described herein are applicable to humans as well as any suitable animal, including, without limitation, dogs, cats, and other pets, as well as rodents, primates, horses, cattle, pigs, etc. The methods can be also applied for clinical research and/or study. As used herein, references to the newborn, neonate, or offspring, etc. expressly refer to the offspring birthed by the pregnant patient receiving the lactoferrin prophylaxis. Additional advantages of the various embodiments of the invention will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments, but is not necessarily included. Thus, the present invention encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### EXAMPLES

The following examples set forth methods in accordance with the invention. It is to be understood, however, that these examples are provided by way of illustration and nothing therein should be taken as a limitation upon the overall scope of the invention.

### Example 1

### Maternal Antepartum Administration of Lactoferrin Ameliorates Neonatal Infection by Bacteremia-Producing Escherichia coli in Mice

E. coli is a major cause of neonatal sepsis. Vaginal pathogenic E. coli strains ascend into the pregnant uterus infecting the offspring of colonized mothers. Lactoferrin (LF) is an antibacterial and immunomodulatory glycoprotein that has been given to preterm newborns after birth to prevent late-onset sepsis. However, the effects of maternal LF administration to prevent neonatal E. coli invasive infection by clinical isolates that produce sepsis and meningitis have not been studied. Neonatal sepsis develops when pathogenic bacteria colonizing the vagina of pregnant women ascend and produce bacteremia and invasive infection in the fetus. Systemic intrapartum administration of antibiotics to pregnant women colonized with group B Streptococcus (GBS) has decreased rates of GBS early-onset sepsis but has resulted in widespread maternal-fetal antibiotic exposure. Maternal GBS antibiotic prophylaxis is not effective against invasive neonatal E. coli strains due to their resistance to multiple antibiotic classes, which continues to increase. There is currently no prevention strategy against neonatal E. coli infection.

The present study was conducted to determine the effects of vaginal lactoferrin administered prenatally to pregnant mice on invasive E. coli disease in their embryos.

### Materials and Methods

Human lactoferrin (LF) 100 μg/mL or placebo were administered vaginally to pregnant C57/BL6 mice twice daily on E16 and E17. Two hours after the 4th dose on E17, mice were infected vaginally with 1×10⁵ colony forming units ts (CFU) of the archetypal bacteremia/meningitis-producing clinical isolate RS218. The bacterial isolate was modified by transposon mutagenesis to constitutively express chloramphenicol (Cam) resistance (RS218-Cam^{R}) as a selection marker. RS218-Cam^{R} shows no growth differences compared to the wild-type strain. On E18, maternal vaginal fluid samples were obtained, and placentas and embryo tissues were collected after humane euthanasia to determine bacterial loads by culture on Cam plates.

### Results

Vaginal RS218-Cam^{R} loads were significantly lower in LF-pretreated pregnant mice compared to placebo, 4.9×10⁶ CFU/mL vs. 1.7×10⁷ CFU/mL, respectively (Welch's t-test P<0.02). Placental RS218-Cam^{R} loads were also lower in LF-pretreated mice compared to placebo, 5.5×103 CFU/100 mg (IQR 1.1×10²-9.6×10⁵, n=16) vs. 2.4×10⁶ CFU/100 mg (IQR 4.3×10⁵-7.6×10⁶, n=17), respectively (Mann-Whitney P<0.001). Bacterial loads in the blood, liver and spleen, and brains of embryos of LF-pretreated dams were significantly lower compared to those of placebo-treated dams (Table1). RS218-Cam^{R} was found in the brain tissue of 50% (total n=16) of embryos from LF-pretreated dams vs. 100% (n=18) of embryo brain tissue in the placebo-pretreated group (Fisher exact test, P<0.001).

TABLE 1

| | LF- | Placebo- | |
|---|---|---|---|
| | pretreatment | pretreatment | P value |
| Blood (median | 433 (0-3100) | 5400 (1600-19666) | <0.01 |
| CFU/mL, IQR) | n = 15 | n = 15 | |
| Liver/spleen (median | 6.6 (0-48) | 1160 (229-4736) | <0.001 |
| CFU/100 mg IQR) | n = 16 | n = 18 | |
| Brain (median | 3.3 (0-56) | 605 (11-2132) | <0.001 |
| CFU/100 mg, IQR) | n = 16 | n = 18 | |

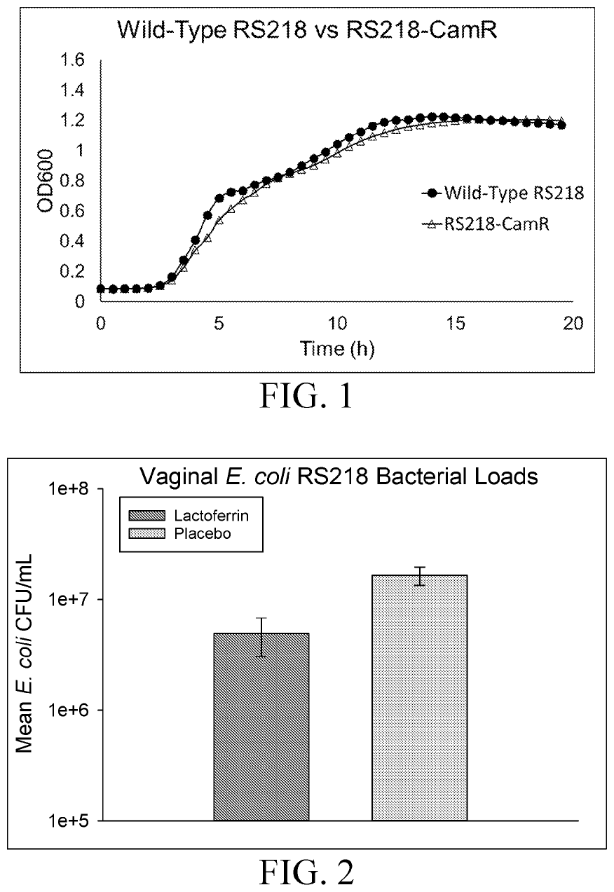

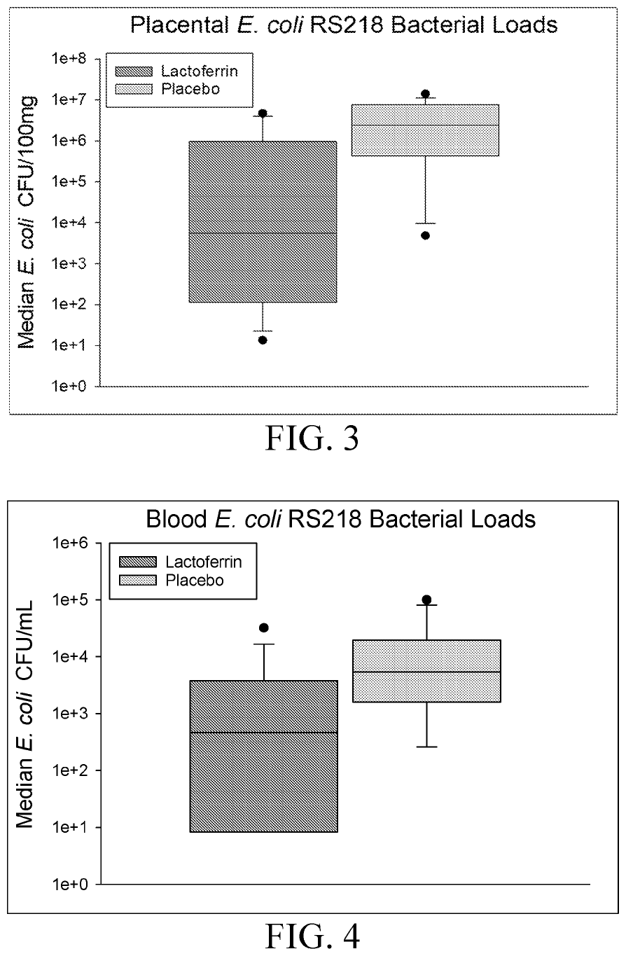

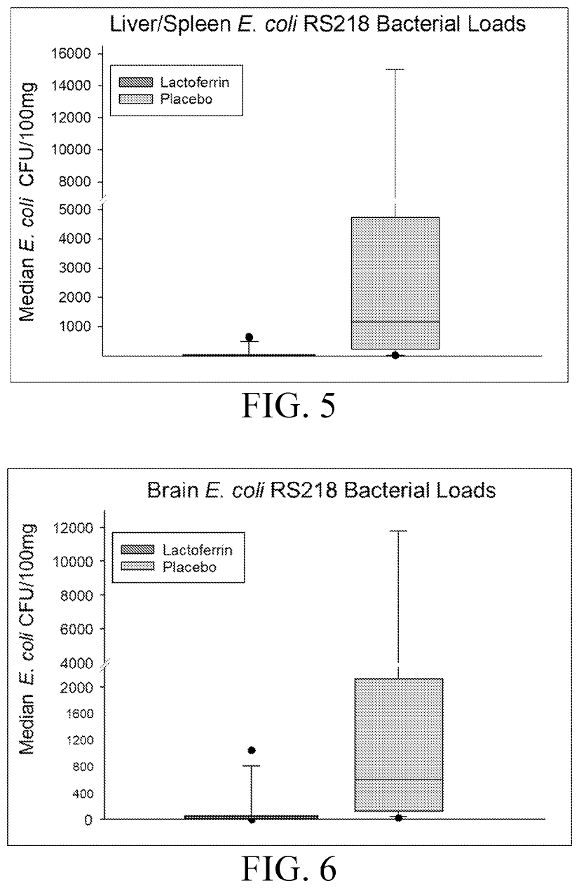

The data are shown in FIGS. 1-6. FIG. 1 shows growth curves in Luria Bertani broth confirming that there are no differences in growth capacity between wild-type RS218 and RS218-Cam^{R}. FIG. 2 shows that vaginal RS218-Cam^{R} loads were significantly lower in LF-pretreated pregnant mice compared to placebo, 4.9×10⁶ CFU/mL vs. 1.7×10⁷ CFU/mL, respectively (Welch's t-test P<0.02). FIG. 3 shows that placental RS218-Cam^{R} loads were also lower in LF-pretreated mice compared to placebo, 5.5×10³ CFU/100 mg (IQR 1.1×10²-9.6×10⁵,n=16) vs. 2.4×10⁶ CFU/100 mg (IQR 4.3×10⁵-7.6×10⁶, n=17), respectively (Mann-Whitney P<0.001). FIG. 4 shows that bacterial loads in the blood of embryos of LF-pretreated dams were significantly lower compared to those of placebo-treated dams, 433 CFU/mL (IQR 0-3100, n=15) vs. 5400 CFU/mL (IQR 1600-19666, n=15), respectively (P<0.01). FIG. 5 shows that bacterial loads in the liver and spleen of embryos of LF-pretreated dams were significantly lower compared to those of placebo-treated dams, 6.6 CFU/100 mg (IQR 0-48, n=16) vs. 1160 CFU/100 mg (IQR 229-4736, n=18), respectively (P<0.001). FIG. 6 shows that bacterial loads in the brain of embryos of LF-pretreated dams were significantly lower compared to those of placebo-treated dams, 3.3 CFU/100 mg (IQR 0-56, n=16) vs. 605 CFU/100 mg (IQR 11-2132, n=18), respectively (P<0.001).

RS218-Cam^{R} was found in the brain tissue of 50% (total n=16) of embryos from LF-pretreated dams vs. 100% (n=18) of embryo brain tissue in the placebo-pretreated group (Fisher exact test, P<0.001).

### CONCLUSIONS

Prenatal maternal vaginal administration of human LF significantly decreased vaginal bacterial loads of a clinically significant neonatal invasive K1+E. coli isolate. Placental bacterial loads, and burden of infection in the offspring were also significantly decreased. The mechanisms by which prenatal LF protects newborns from invasive E. coli disease is still unclear. Prenatal lactoferrin is a potential preventative intervention against neonatal sepsis.

### Example 2

### Studies Conducted Using Mice and Bovine Lactoferrin

In Example 1, we have demonstrated the effectiveness of human lactoferrin at 100 mcg/mL in the animal model. Human lactoferrin availability is extremely limited making its use unfeasible for large scale applications. Bovine lactoferrin is structurally and functionally similar, and is used in humans, including newborns, for other indications.

In these studies, bovine lactoferrin was tested in the animal model at concentrations of 0.1 mg/mL and 10 mg/mL to determine the optimal concentration range to maximize lactoferrin's protective effect. Fifty microliters of bovine lactoferrin solution at different concentrations are administered vaginally twice daily to pregnant C57BL/6 mice starting on the 16th day of pregnancy, or E16 (total gestation duration is approximately 19-21 days). After 48 h of bovine lactoferrin pre-treatment, mice are inoculated vaginally once with approximately 100,000 colony-forming units (CFU) of neonatal E. coli strain RS218. On E18, mice are euthanized and samples from dams (blood, serum, cervix, spleen, and vaginal lavage) and embryos (placenta, brain, liver/spleen, blood) are collected for bacterial and lactoferrin quantification.

In separate experiments, twice daily prophylactic lactoferrin administration will be continued until spontaneous delivery occurs.

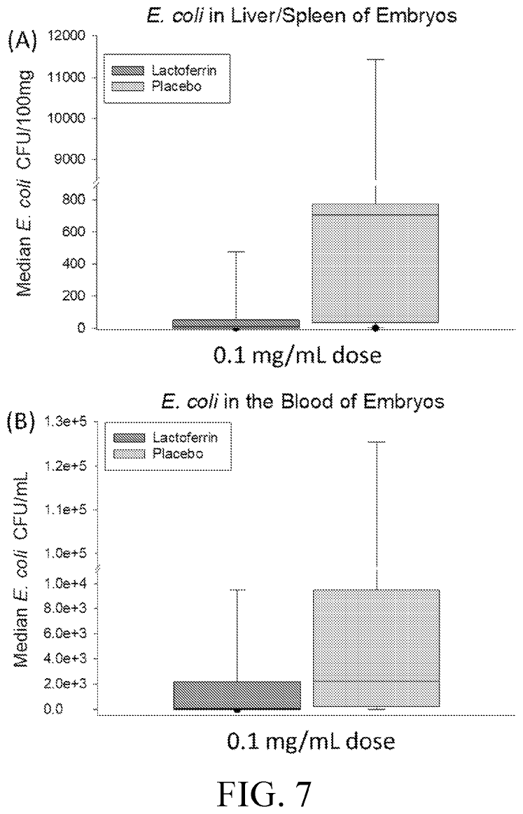

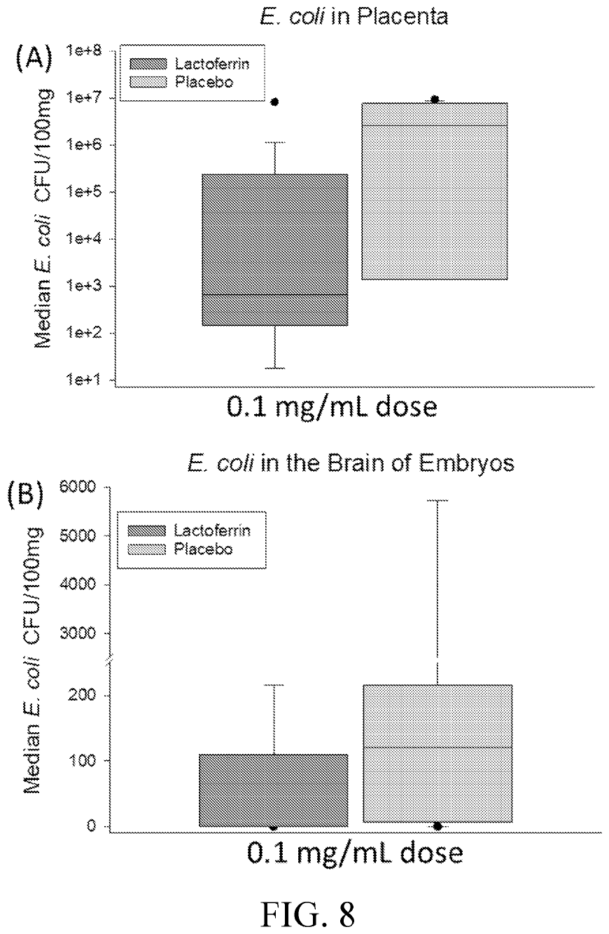

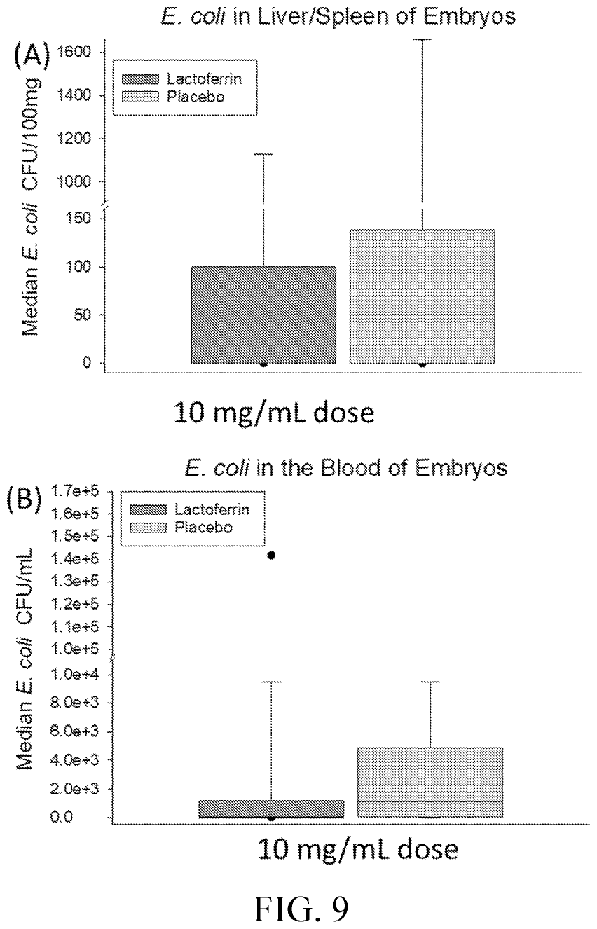

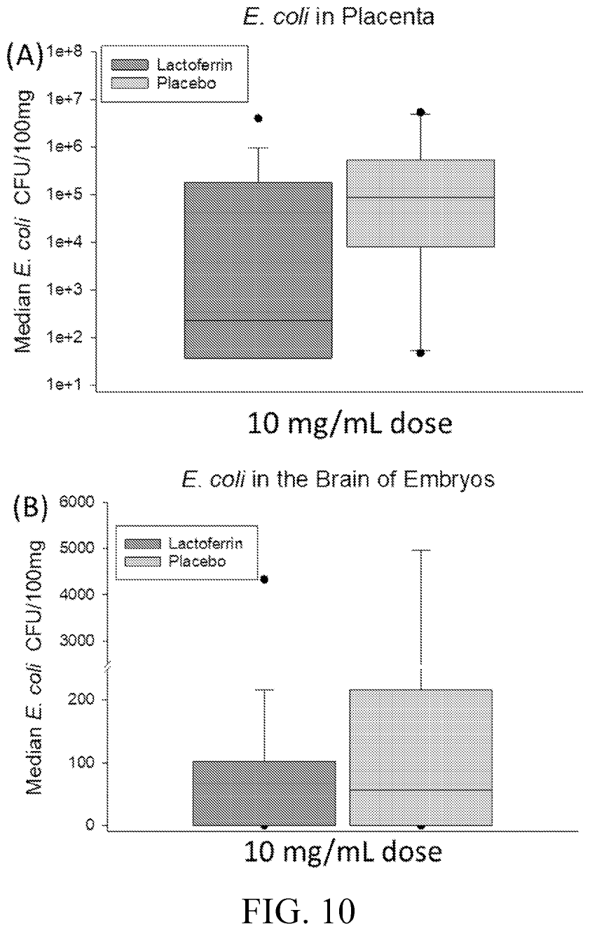

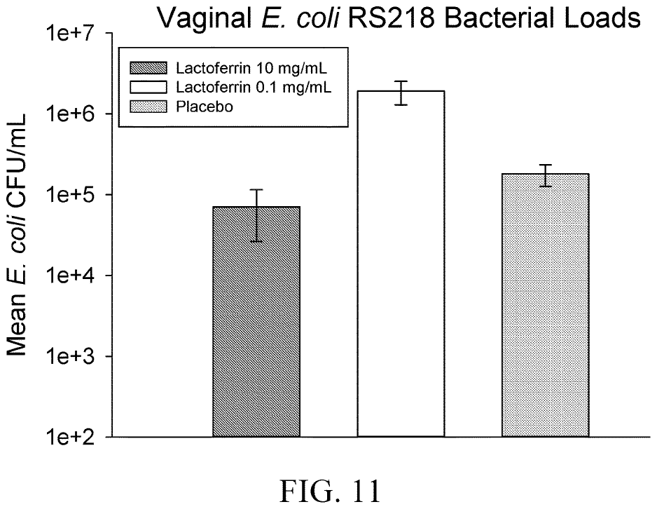

The data in FIGS. 7-11 shows the results from the two dosages of bovine lactoferrin tested, 0.1 mg/mL and 10 mg/mL. The results look similar to those in Example 1. For example, the bacterial loads in the placentas at both dosages were significantly decreased by pretreatment with BLF. Notably, however, as shown in FIG. 11, the bovine LF administration does not seem to significantly decrease the vaginal bacterial load in the treated dams; however, the bacterial load in the placentas and embryos themselves is significantly decreased as shown in FIGS. 7-10. The data thus confirmed the effects of bovine LF on protecting the embryos from infection differs from human LF in decreasing maternal vaginal bacterial loads. This could be because LF prevents bacteria from ascending into the uterine cavity by “trapping” it in the vagina but not necessarily stopping it from growing in the vaginal fluid. These effects may be different between the use of human vs. bovine lactoferrin.

In separate experiments, the mice are infected with E. coli first and then treated with bLF.

1. A method of decreasing bacterial loads in a neonate to prophylactically treat neonatal E. coli infection during pregnancy, said method comprising:
vaginally administering an effective amount of lactoferrin to a pregnant mammal during the second half of gestation and before birth of said neonate,
wherein bacterial load of E. coli in said neonate is reduced after birth, as compared to an untreated control.

2. The method of claim 1, wherein said lactoferrin is administered at least once daily.

3. The method of claim 2, wherein said lactoferrin is administered daily for at least 7 days before birth.

4. The method of claim 2, wherein said lactoferrin is administered twice daily.

5. The method of claim 1, wherein said lactoferrin is administered 24 hours or less before onset of labor in said pregnant mammal.

6. The method of claim 1, wherein said lactoferrin is administered beginning seven days before a calculated due date for birth of said neonate.

7. The method of claim 1, wherein said lactoferrin is administered at least 12 hours before onset of labor.

8. The method of claim 1, wherein said lactoferrin is administered to said pregnant mammal after onset of labor and before birth of said neonate.

9. The method of claim 1, wherein said lactoferrin is administered at least once after onset of labor in said pregnant mammal and before birth of said neonate.

10. The method of claim 1, wherein said lactoferrin is administered in a dosage amount and frequency that maintains greater than about 10 μg/mL lactoferrin in the vaginal fluid of said pregnant mammal.

11. The method of claim 1, wherein said administering step comprises applying the lactoferrin to mucosal membranes in the vagina of the pregnant mammal.

12. The method of claim 1, wherein said administering step comprises applying a composition comprising said lactoferrin dispersed or suspended in a pharmaceutically acceptable carrier to said pregnant mammal.

13. The method of claim 12, wherein said composition is a liquid solution, cream, lotion, ointment, salve, hydrogel, thin film, suppository, dry powders, dissolving tablets, patches, capsules, and the like in which said lactoferrin is dispersed or suspended.

14. The method of claim 13, said composition comprises from about 100 μg/mL lactoferrin to about 100 mg/mL lactoferrin, preferably from about from about 100 μg/mL to about 10 mg/mL lactoferrin, preferably from about 100 μg/mL to about 1,000 μg/mL.

15. The method of claim 1, wherein said administering step comprises inserting a bolus, hydrogel, thin film, or suppository comprising said lactoferrin into the vagina of the pregnant mammal.

16. The method of claim 1, wherein bacterial loads are not necessarily significantly decreased in said pregnant mammal after said administering step as compared to an untreated control.

17. A prophylactic vaginal formulation for maternal administration during pregnancy to decrease bacterial loads in a neonate and inhibit or ameliorate neonatal E. coli invasive infection including sepsis, said composition comprising lactoferrin dispersed or suspended in a pharmaceutically acceptable carrier.

18. The formulation of claim 17, wherein said composition is selected from the group consisting of a liquid solution, cream, lotion, ointment, salve, hydrogel, thin film, suppository, dry powders, dissolving tablets, patches, capsules, and the like, in which said lactoferrin is dispersed or suspended.

19. Use of a prophylactic vaginal formulation according to claim 17 to prepare a vaginal medicament for maternal administration, preferably during second half of pregnancy, to decrease bacterial loads in a neonate and inhibit neonatal E. coli invasive infection including sepsis.

20. Use of a prophylactic vaginal formulation according to claim 17 for maternal vaginal administration, preferably during the second half of pregnancy, to prophylactically treat neonatal E. coli infection.
